(19) United States
(12) Patent Application Publication
(10) Pub. No.: US 2015/0359817 A1
(43) Pub. Date: Dec. 17, 2015

(54) METHOD OF TREATING INFLAMED, IRRITATED, AND SENSITIVE SKIN USING A POLYLMER SUSPENSION SYSTEM, METHOD FOR PREPARING THE SAME, AND PRODUCTS MADE THEREOF

(71) Applicants: George Costa, Hertfordshire (GB); Tony Wilson, Hertfordshire (GB)

(72) Inventors: George Costa, Hertfordshire (GB); Tony Wilson, Hertfordshire (GB)

(73) Assignee: Dreamskin Health Limited, Hatfield (GB)

(21) Appl. No.: 14/306,873

(22) Filed: Jun. 17, 2014

(51) Int. Cl. A61K 31/785; A61K 45/06

(52) U.S. Cl. CPC A61K 31/785; A61K 45/06

(57) ABSTRACT

One aspect of the present invention provides a use of polymer 2-(methacryloyloxy)ethyl-2-(trimethylammonio)ethyl phosphate-stearylmethacrylate copolymer suspension as a compound which is beneficial to inflamed, irritated and sensitive skin. According to an alternative further aspect of the present invention there is provided a method of preparing a fabric which is non-irritating to the skin, said method comprising: scouring a fabric; rinsing the scoured fabric; coating the fabric with a polymer suspension having dual hydrophilic and hydrophobic characteristics; and finally setting the coated fabric to the required dimensions using a heat drying process so that an original handfeel of the fabric is not affected by the coating.

### BACKGROUND OF THE INVENTION

1. Field of the Invention

This invention relates to the use of a polymer which can be beneficial to the skin. In particular it is directed towards the use of the polymer on fabrics and garments made of said fabrics. It can be used to help retain moisture in the skin and act as a barrier to irritants.*

2. Description of the Related Art

Inflamed, irritated and sensitive skin is a significant problem which may result from a number of causes such as eczema, atopic dermatitis and dry skin. There is therefore an ongoing need to look for ways of treating and controlling this, providing relief from the condition by soothing the problem areas and, ultimately, leading to recovery. Many of the known treatments require compliance of the user to follow a specified medical treatment regime, for example in the use of emollients or topical steroids. Non-compliance can cause problems and regression of the condition.

Further, standard items of clothing can aggravate and exacerbate conditions, particularly in problem areas such as under the arm, in the elbow and behind the knee. The clothing may retain irritants such as pollen and washing powder residues which are then placed next to the inflamed, irritated or sensitive skin and the symptoms persist or even get worse.

As a result, the present invention is therefore directed towards the use of a polymer which can be beneficial to the skin to help control the loss of moisture from damaged skin and thereby provide relief from itching associated with dry skin conditions. The polymer may be delivered by items of clothing which ensures the benefits of the polymer are delivered simply and effectively to the skin.

### ASPECTS AND SUMMARY OF THE INVENTION

In response, it is now recognized that there is a need for an improved use and method of use for a polymer beneficial to inflamed, irritated, and sensitive skin.

According to one alternative and adaptive embodiment of the present invention, there is provided the use of the polymer 2-(methacryloyloxy)ethyl-2-(trimethylammonio)ethyl phosphate-stearylmethacrylate copolymer suspension (understood as Dreamskin polymer, Dreamskin™ is a Trademark (™) of Dreamskin Health Limited) as a compound which is beneficial to inflamed, irritated and sensitive skin.

The polymer suspension may be used neat, or it may be further diluted with water or other components. In one embodiment, the polymer suspension is diluted in water such that the quantity of polymer suspension is in the range 50-200 g/l, or in the range 75-150 g/l, or in the range 80-1200, or in the range 90-110 g/l.

The polymer suspension may be applied to a fabric. The polymer suspension may be applied by any suitable means such as dyeing, spraying, coating using a roller or padding. The fabric may then be used in the manufacture of clothing. The fabric used to carry the polymer suspension may be any suitable material such as silk, cotton, linen, polyester, polypropylene nonwoven (carded, spun or air-laid bonded) or wool. In an embodiment, the fabric is a silk fabric stripped or serecin to a point below a level which could cause skin sensitisation, thus rendering the fabric medical-grade silk fabric.

It will be understood that the word serecin is well known to those of skill in the art and is flexibly employed in a descriptive sense. Degumming is the process of removing the sericin, a sticky substance produced by the silkworm that holds the strands of silk together. It is also known as silk scouring. Removing the gum improves the lustier, color, hand, and texture of the silk. The degumming waste liquor that is rich in sericin content is being used as a raw material for the production of sericin powder. The sericin powder is being used in the cosmetic industry as moisturizer, in hair-care products and also as a sustainable natural textile finish. Removal of sericin from the waste degumming liquor also substantially reduces the effluent. Discussions regarding serecin (or sericin) can be further considered by study of the Handbook of Textile Fibers, J. Gordon Cook, Vol: 1, regarding Natural fibers, or the article Degumming of silk fabric with several proteases, by Giuliano Freddia, Raffaella Mossottib, Riccardo Innocentib, Journal of Biotechnology, Volume 106, Issue 1, 5 Dec. 2003, Pages 101-112.

Such polymer coated medical-grade silk clothing reduces the unregulated loss of moisture from damaged skin and helps to regulate and normalise body temperature. As a consequence of its very low friction coefficient, the polymer coated garments will not adhere to weeping eczema lesions and also protects the wearer's skin by acting as a barrier to common irritants such as pollen and washing powder residues.

Such clothing is intended to offer symptomatic relief from itching associated with dry skin conditions, soothing dry and damaged skin and providing accelerated relief and recovery for sufferers of contact dermatitis and eczema. These benefits are delivered simply whilst wearing the garments and do not rely on patient compliance as is needed for more common treatments.

The clothing manufactured using the fabric coated with the Dreamskin polymer suspension is suitable for wearing as a barrier to irritants and/or for retaining moisture in the skin. The fabric may optionally also be treated with an antimicrobial material. The clothing optionally is prepared with flat, offset seams to minimize seam rubbing in areas which may be, or become, sore or irritated and aggravated by normal hemmed seams.

In alternative and non-limiting embodiment of the present invention, the fabric is a serecin free, medical grade silk yarn coated with the DreamSkin polymer suspension during exhaust dyeing. The polymer suspension attaches to the fabric by ionic-coupling when in a mildly acidic solution, which creates a firm and wash durable bond between the fibres and the polymer. During the final drying step of the manufacturing process which follows any of the coating methods, the polymer molecules reorder into a lamella structure which resembles the structure of normal skin and it is this reordering which helps to retain moisture in the skin.

The multilayer lamella structure formed has a very low friction coefficient. This low friction coefficient element provides an external barrier effect against irritants (effectively resisting foreign particles from either attaching to the fabric during laundering or from dispersing through the fabric) and the hydrophobic element provides a moisture retaining property.

The clothing manufactured using the coated fabric may be of a form fitting ergonomic design, which is close fitting but comfortable to wear in close contact with the skin. The clothing can be worn alone or as undergarments.

According to a further aspect of the present invention there is provided a method of preparing a fabric which is non-irritating to the skin, said method comprising: scouring a fabric; rinsing the scoured fabric; coating the fabric with a polymer suspension having dual hydrophilic and hydrophobic characteristics; and finally setting the coated fabric to the required dimensions using a heat drying process so that the original handfeel of the fabric is not affected by the coating.

The fabric may be selected from any suitable material such as silk, cotton, linen, polyester, polypropylene nonwoven (either carded, spun or air-laid bonded) or wool. In one embodiment the fabric may be silk fabric stripped of serecin to a point below a level which could cause skin sensitisation thus rendering it medical-grade silk fabric.

The scouring may comprise any suitable steps to ensure the fabric is substantially clear of irritants and impurities. In one embodiment, the scouring may comprise exhaustion scouring with a suitable solvent. The scouring step must be vigorous enough to clean the fabric, but must be gentle enough to avoid fabric abrasion damage. The scouring step may take place at an elevated temperature, for example at a temperature above 30° C., such as in the range 30-120° C., or in the range 40-120° C., or in the range 60-110° C., or in the range 70-100° C. or in the range 80-95° C. The scouring step may last for in excess of 10 minutes, for example for a time in the range 10-120 minutes, or in the range 10-90 minutes, or in the range 15-75 minutes, or in the range 15-60 minutes, or in the range 20-40 minutes.

The liquor (composition) used to scour the fabric may additionally be drained before rinsing. The used solvent may be collected and rejuvenated and then recycled. In the embodiment where the fabric is made from a silk yarn, the scoured fabric should be virtually free of serecin.

The rinsing step may be a single step at either ambient or elevated temperature, or it may be a multi-stage process at the same or different temperatures. In one embodiment, the rinsing step may comprise a two stage process where the scoured fabric is first rinsed at an elevated temperature and drained, and then rinsed at ambient temperature. The first rinse stage may be at a temperature above 30° C., for example in the range 30-60° C., or in the range 30-50° C., or in the range 35-45° C. The second rinse stage may be at a temperature in the range 5-25° C., or in the range 10-20° C.

The rinse stages may have equal or different lengths. The first stage warm rinse may be for 10 minutes or more, for example in the range 10-30 minutes, or in the range 10-20 minutes, or in the range 10-15 minutes. The second stage cold rinse may be for up to 10 minutes, for example in the range 1-10 minutes, or in the range 2-8 minutes, or in the range 3-7 minutes.

The rinse stage or stages will generally be carried out using water, but the water may have chemicals added to it to adjust the pH as necessary. For example, the liquid for the second rinse stage may have an acid, such as acetic or citric acid, added to it to reduce the pH. The pH may be adjusted to be in the range 3.5-8, or in the range 4.5-7.5, or in the range 5.5-7, or in the range 6-7.

The drying step may be by any suitable means. In one embodiment the drying is tension free to avoid or minimize stretching or bagging damage to the fabric. For example the drying may be in a tensionless net conveyer.

The coating step may be by any suitable means such as during the dyeing process, spraying, coating using a kiss roller or padding. In one embodiment of the present invention, the polymer is applied by chemical padding.

The polymer suspension to be applied has dual hydrophilic and hydrophobic characteristics and may be the Dreamskin polymer (TM, Dreamskin Health Limited). The active ingredient may be 2-(methacryloyloxy)ethyl-2-(trimethylammonio)ethyl phosphate-stearylmethacrylate copolymer. The polymer suspension may be applied neat or it may be diluted with water and other components. In one embodiment the polymer suspension is diluted in water such that the quantity of polymer suspension is in the range 50-200 g/l, or in the range 75-150 g/l, or in the range 80-120 g/l, or in the range 90-110 g/l per kilogram of fabric.

The final drying step may be carried out by any suitable means such as air drying at elevated temperature. The drying step may be at a temperature of 60° C. or above, for example in the range 60-140° C., or in the range 75-140° C., or in the range 85-130° C., or in the range 90-125° C., or in the range 100-120° C. The drying step may have a duration from 30 seconds or longer, for example 30-300 seconds, or 50-250 seconds, or 75-200 seconds, or 80-160 seconds, or 85-120 seconds.

According to one alternative aspect of the present invention, there is provided a use of 2-(methacryloyloxy)ethyl-2-(trimethylammonio)ethyl phosphate-stearylmethacrylate copolymer suspension as a compound which is beneficial to inflamed, irritated and sensitive skin.

According to another alternative aspect of the present invention, the polymer suspension is used in diluted form.

According to another alternative aspect of the present invention, the polymer suspension is diluted with water.

According to another alternative aspect of the present invention, the diluted polymer is further mixed with additional compounds.

According to another alternative aspect of the present invention, the additional compound is an antimicrobial agent.

According to another alternative aspect of the present invention, the polymer suspension is diluted with water such that the quantity of polymer suspension is in the range 50-200 g/l.

According to another alternative aspect of the present invention, the polymer suspension is diluted with water such that the quantity of polymer suspension is in the range 75-150 g/l.

According to another alternative aspect of the present invention, the polymer suspension is diluted with water such that the quantity of polymer suspension is in the range 80-120 g/l.

According to another alternative aspect of the present invention, the polymer suspension is diluted with water such that the quantity of polymer suspension is in the range 90-110 g/l.

According to another alternative aspect of the present invention, the polymer suspension is applied to a fabric.

According to another alternative aspect of the present invention, the polymer suspension is applied by dyeing, spraying, coating using a roller or padding.

According to another alternative aspect of the present invention, the fabric is selected from silk, cotton, linen, polyester, polypropylene, or wool.

According to another alternative aspect of the present invention, the fabric is a serecin free, medical grade silk.

14. A use as claimed in claim 10, in which the fabric is used in the manufacture of clothing suitable for wearing as a barrier to irritants and/or for retaining moisture in the skin.

According to another alternative aspect of the present invention, the clothing is manufactured with flat, offset seams to minimize seam rubbing.

According to another alternative aspect of the present invention, the clothing is of a form fitting ergonomic design which is close fitting to the skin.

According to another alternative aspect of the present invention, there is provided a method of preparing a fabric which is beneficial to inflamed, irritated and sensitive skin, said method comprising: scouring a fabric; rinsing the cleaned fabric; coating the fabric with a polymer suspension having dual hydrophilic and hydrophobic characteristics; and setting the coated fabric to the required dimensions using a heat drying process.

According to another alternative aspect of the present invention, there is provided a method in which the fabric is selected from silk, cotton, linen, polyester, polypropylene nonwoven (carded, spun or air laid) or wool.

According to another alternative aspect of the present invention, there is provided a method in which the fabric is medical-grade silk fabric.

According to another alternative aspect of the present invention, there is provided a method in which the scouring step comprises exhaustion scouring with a suitable solvent.

According to another alternative aspect of the present invention, there is provided a method in which the solvent used for the scouring step is selected from a blend of surfactants, soda ash and sequestrants.

According to another alternative aspect of the present invention, there is provided a method in which the scouring step is at an elevated temperature

According to another alternative aspect of the present invention, there is provided a method in which the temperature is above 30° C.,

According to another alternative aspect of the present invention, there is provided a method in which the temperature is in the range 30-120° C.

According to another alternative aspect of the present invention, there is provided a method in which the temperature is in the range 40-120° C.

According to another alternative aspect of the present invention, there is provided a method in which the temperature is in the range 60-110° C.

According to another alternative aspect of the present invention, there is provided a method in which the temperature is in the range 70-100° C.

According to another alternative aspect of the present invention, there is provided a method in which the temperature is in the range 80-95° C.

According to another alternative aspect of the present invention, there is provided a method in which the scouring step lasts for in excess of 10 minutes.

30. A method as claimed in claim 29, in which the scouring step lasts for a time in the range 10-120 minutes.

According to another alternative aspect of the present invention, there is provided a method in which the scouring step lasts for a time in the range 10-90 minutes.

According to another alternative aspect of the present invention, there is provided a method in which the scouring step lasts for a time in the range 15-75 minutes.

According to another alternative aspect of the present invention, there is provided a method in which the scouring step lasts for a time in the range 15-60 minutes.

According to another alternative aspect of the present invention, there is provided a method in which the scouring step lasts for a time in the range 20-40 minutes.

According to another alternative aspect of the present invention, there is provided a method in which the scoured fabric is additionally drained before rinsing.

According to another alternative aspect of the present invention, there is provided a method in which the rinsing step is a single step.

According to another alternative aspect of the present invention, there is provided a method in which the rinsing step is at ambient or elevated temperature

According to another alternative aspect of the present invention, there is provided a method in which the rinsing step is a multi-stage process.

According to another alternative aspect of the present invention, there is provided a method in which the multiple stages are at the same temperatures.

According to another alternative aspect of the present invention, there is provided a method in which the multiple stages are at different temperatures.

According to another alternative aspect of the present invention, there is provided a method in which the rinsing step comprise a two stage process.

According to another alternative aspect of the present invention, there is provided a method in which the two stage process comprises a first stage rinse at an elevated temperature and a second stage rinse at ambient temperature.

According to another alternative aspect of the present invention, there is provided a method in which the first rinse stage is at a temperature above 30° C.

According to another alternative aspect of the present invention, there is provided a method in which the temperature is in the range 30-60° C.

According to another alternative aspect of the present invention, there is provided a method in which the temperature is in the range 30-50° C.

According to another alternative aspect of the present invention, there is provided a method in which the temperature is in the range 35-45° C.

According to another alternative aspect of the present invention, there is provided a method in which the second rinse stage is at a temperature in the range 5-25° C.

According to another alternative aspect of the present invention, there is provided a method in which the temperature is in the range 10-20° C.

According to another alternative aspect of the present invention, there is provided a method in which the first and second rinse stages have an equal length.

According to another alternative aspect of the present invention, there is provided a method in which the first and second rinse stages have different lengths.

According to another alternative aspect of the present invention, there is provided a method in which the first stage warm rinse has a duration of 10 minutes or more.

According to another alternative aspect of the present invention, there is provided a method in which the duration is in the range 10-30 minutes.

According to another alternative aspect of the present invention, there is provided a method in which the duration is in the range 10-20 minutes.

According to another alternative aspect of the present invention, there is provided a method in which the duration is in the range 10-15 minutes.

According to another alternative aspect of the present invention, there is provided a method in which the second stage cold rinse has a duration of up to 10 minutes.

According to another alternative aspect of the present invention, there is provided a method in which the duration is in the range 1-10 minutes.

According to another alternative aspect of the present invention, there is provided a method in which the duration is in the range 2-8 minutes.

According to another alternative aspect of the present invention, there is provided a method in which the duration is in the range 3-7 minutes.

According to another alternative aspect of the present invention, there is provided a method in which the rinse stage uses water.

According to another alternative aspect of the present invention, there is provided a method in which the rinse stage additionally has chemicals added to it to adjust the pH.

According to another alternative aspect of the present invention, there is provided a method in which the rinse stage has acetic acid or citric acid added.

According to another alternative aspect of the present invention, there is provided a method in which the pH is adjusted to be in the range 3.5-8.

According to another alternative aspect of the present invention, there is provided a method in which the pH is adjusted to be in the range 5.5-7.

According to another alternative aspect of the present invention, there is provided a method in which the pH is adjusted to be in the range 6-7.

According to another alternative aspect of the present invention, there is provided a method in which the drying step is tension free to avoid or minimize damage to the fabric.

According to another alternative aspect of the present invention, there is provided a method in which the coating step is by lamination, spraying, during the dyeing process, rolling with a kiss roller or padding.

According to another alternative aspect of the present invention, there is provided a method in which the coating step is chemical padding.

According to another alternative aspect of the present invention, there is provided a method in which the polymer suspension applied is 2-(methacryloyloxy)ethyl-2-(trimethylammonio)ethyl phosphate-stearylmethacrylate copolymer suspension.

According to another alternative aspect of the present invention, there is provided a method in which the polymer suspension is applied neat to the fabric.

According to another alternative aspect of the present invention, there is provided a method in which the polymer suspension is applied to the fabric diluted with water and/or other components.

According to another alternative aspect of the present invention, there is provided a method in which the polymer suspension is diluted in water such that the quantity of polymer suspension is in the range 50-200 g/l per kg of fabric.

According to another alternative aspect of the present invention, there is provided a method in which the quantity of polymer suspension is in the range 75-150 g/l per kg of fabric.

According to another alternative aspect of the present invention, there is provided a method in which the quantity of polymer suspension is in the range 80-120 g/l per kg of fabric.

According to another alternative aspect of the present invention, there is provided a method in which the quantity of polymer suspension is in the range 90-110 g/l per kg of fabric.

According to another alternative aspect of the present invention, there is provided a method in which the final drying step is air drying at elevated temperature.

According to another alternative aspect of the present invention, there is provided a method in which the drying step is at a temperature of 60° C. or above.

According to another alternative aspect of the present invention, there is provided a method in which the temperature is in the range 60-140° C.

According to another alternative aspect of the present invention, there is provided a method in which the temperature is in the range 75-140° C.

According to another alternative aspect of the present invention, there is provided a method in which the temperature is in the range 85-130° C.

According to another alternative aspect of the present invention, there is provided a method in which the temperature is in the range 90-125° C.

According to another alternative aspect of the present invention, there is provided a method in which the temperature is in the range 100-120° C.

According to another alternative aspect of the present invention, there is provided a method in which the drying step has a duration from 30 seconds or longer.

According to another alternative aspect of the present invention, there is provided a method in which the duration is from 30-300 seconds.

According to another alternative aspect of the present invention, there is provided a method in which the duration is from 50-250 seconds.

According to another alternative aspect of the present invention, there is provided a method in which the duration is from 75-200 seconds.

According to another alternative aspect of the present invention, there is provided a method in which the duration is from 80-160 seconds.

According to another alternative aspect of the present invention, there is provided a method in which the duration is from 85-120 seconds.

According to another alternative aspect of the present invention, there is provided a method of using coated fabric in the manufacture of clothing suitable for wearing as a barrier to irritants and/or for retaining moisture in the skin.

According to another alternative aspect of the present invention, there is provided a method of using coated fabric in the manufacture of clothing in which the clothing is manufactured with flat offset seams.

According to another alternative aspect of the present invention, there is provided a method of using coated fabric in the manufacture of clothing in which the fabric is a serecin free, medical grade silk fabric coated with Dreamskin polymer.

According to another alternative aspect of the present invention, there is provided a method of using coated fabric in the manufacture of clothing in which the clothing is of a form fitting ergonomic design which is close fitting to the skin.

According to another alternative aspect of the present invention, there is provided a method of using coated fabric in the manufacture of clothing in which clothing is selected from long sleeved and short sleeved tops, leggings, briefs, boxer briefs, hosiery, head masks, eye masks, tubular sleeves, bodysuits, pajamas and gloves.

According to another alternative aspect of the present invention, there is provided a garment manufactured from serecin free, medical grade silk coated with Dreamskin polymer.

According to another alternative aspect of the present invention, there is provided a garment selected from long sleeved and short sleeved tops, leggings, briefs, boxer briefs, hosiery, head masks, eye masks, tubular sleeves, bodysuits, pajamas and gloves.

The above and other aspects, features, steps, uses, and advantages of the present invention will become apparent from the following description read in conjunction with the accompanying drawings, in which like reference numerals designate the same elements.

### BRIEF DESCRIPTION OF THE DRAWINGS

None

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

According to one adaptive embodiment of the invention, a fabric made from serecin free (medical-grade) silk yarn is treated and coated with DreamSkin polymer suspension to produce a fabric which hydrates the skin, is non-irritating to the skin and can be used to cover skin which is dry, inflamed, irritated or sensitive. Medical-grade silk is highly absorbent, capable of absorbing up to 30% of its weight in moisture without feeling damp. The treated fabric can be manufactured into clothing which can be close fitting but which is completely breathable and does not act as an occlusive barrier.

The medical-grade silk yarn is first visually inspected to check that there are no signs of wear or imperfections. It is then cleaned using a scouring step in which a scouring solution (such as Scour LB, comprising an intimate blend of surfactants, soda ash and sequestrants) is applied at a dilution of 1 to 20 or I to 30 for 20-30 minutes at a temperature of 90° C. The scouring is conducted in a long nose tensionless soft flow high temperature dyeing to avoid fabric abrasion damage. The solution becomes slightly alkaline and this should be monitored to ensure that the pH does not go above 8.

The scoured fabric is then drained and the solution may be treated such that the chemicals can be rejuvenated and recycled. The level of serecin now present in the fabric is now below a level which could cause skin sensitization (medical grade).

The scoured fabric is then treated to a warm rinse at a temperature of 40° C. for 10 minutes and then drained. The fabric is then treated to a cold rinse for 5 minutes and drained. The solution for the cold rinse includes a quantity of either acetic or citric acid to bring the pH of the rinse solution to a range between 3.5-6.

In preparation for the coating with the polymer suspension by chemical padding, a mixture is prepared as follows: The Dreamskin polymer suspension is diluted with water to a ratio between 1-5 to 1-10 and the mixture is transferred to the padding bath for the coating step.

When the fabric is coated by means of chemical padding, the dosage range is 50-120 gm of Dreamskin per kilo of fabric (100 gm/kilo as standard) and the fabric is subjected to 1 dip/1 nip at 80% pick up in the prepared mixture of Dreamskin. The speed is kept deliberately slow to allow the chemicals to penetrate into the fabrics. After treatment every individual strand of silk in the fabric is coated with the polymer.

When applying the Dreamskin polymer suspension during exhaust dyeing, the dosage is between 50-200 ml of Dreamskin per kilo of fabric (120 gm/kil0 as standard) and a liquor ratio of 1-10 at a pH range of 3.5-4.5 at 40-50° C. for 20 minutes.

The coated fabric is then dried at 110° C. for 90 seconds or 100° C. for 120 seconds. The fabric is set to the original length and width and air dried. If necessary in order to reduce subsequent shrinkage the fabric may then be sanforized® in accordance with normal practice. Sanforidized® is a preshrinking (of a fabric) and fabric of the same or clothing, and a trademark of GTB Holdings Corp. (DEL) of New York, N.Y., and is known to those of skill in the art.

The coated fabric may then be used to manufacture a range of clothing garments which can be worn either alone or as undergarments. The clothing is typically close fitting and is prepared with flat, offset seams to minimize seam rubbing in areas of the body which are already, or are prone to become, sore and irritated. These areas typically include under the arm, the inner elbow, at the back of the knee.

The clothes made from the coated fabric are comfortable to wear and help the skin to retain moisture, thereby reversing the effects of dry, irritated skin, reducing itch and helping the skin to heal. The clothes also repel irritants by acting as a barrier against common irritants such as washing powder residue and pollen, thereby preventing further irritation of the skin.

The fabric may be made into a range of clothing including, but not limited to, long sleeved and short sleeved tops, leggings, briefs, boxer briefs, hosiery, head masks, eye masks, tubular sleeves, bodysuits, pajamas and gloves. These may be for men and women and for children.

Having described at least one of the preferred embodiments of the present invention, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

1. A method of treating inflamed, irritated, and sensitive skin, comprising the steps of:
applying polymer a suspension to the skin; and
said polymer suspension comprising 2-(methacryloyloxy)ethyl-2-(trimethylammonio)ethyl phosphate-stearylmethacrylate copolymer.

2. The method, according to claim 1, wherein:
said polymer suspension further comprises antimicrobial agent.

3. The method, according to claim 2, wherein:
said polymer suspension is diluted with water such that the quantity of polymer suspension is in the range 50-2000.

4. The method, according to claim 3, wherein:
before said step of applying to the skin, said the polymer suspension is applied to a fabric.

5. The method, according to claim 4, wherein:
said fabric is selected from a group consisting of silk, cotton, linen, polyester, polypropylene, wool, and a combination of the same.

6. The method, according to claim 5, wherein:
said fabric is a serecin free silk.

7. (canceled)

8. (canceled)

9. (canceled)

10. (canceled)

11. (canceled)

12. (canceled)

13. (canceled)

14. (canceled)

15. (canceled)

16. (canceled)
